(19) United States
(12) Patent Application Publication
(10) Pub. No.: US 2024/0148447 A1
(43) Pub. Date: May 9, 2024

(54) Systems And Methods For Associating Components Of A Surgical Instrument For Navigation-Assisted Surgery

(71) Applicant: Stryker European Operations Limited, Carrigtwohill, Co Cork (IE)

(72) Inventors: Richard Evan Stein, Flower Mound, TX (US); Prashanth Chetlur Adithya, Kalamazoo, MI (US); Kyle David Hanis, Galesburg, MI (US); Alison Paige Kuhn, Kalamazoo, MI (US); Zachary Bolthouse, Kalamazoo, MI (US)

(73) Assignee: Stryker European Operations Limited, Carrigtwohill, Co Cork (IE)

(21) Appl. No.: 18/549,147

(22) PCT Filed: Mar. 7, 2022
(86) PCT No.: PCT/IB2022/052012

§ 371 Date: Sep. 5, 2023

Related U.S. Application Data
(60) Provisional application No. 63/157,321, filed on Mar. 5, 2021.

(51) Int. Cl. A61B 34/20; A61B 34/00

(52) U.S. Cl. CPC A61B 34/20; A61B 34/25; A61B 2034/252

(57) ABSTRACT

Systems and methods of associating components of a surgical instrument for navigation-assisted surgery, and controlling the surgical instruments. A first association is generated between the end effector and the navigation array from an input based on an arrangement of tracking elements of the navigation array, and an identification signal. A second association is generated between a power source and the navigation array from a battery signal wirelessly transmitted from the power source that is indicative of current being drawn on the power source. The power source may be controlled based on a position and/or an orientation of the end effector and a pose of a patient tracker. Should the position of the end effector cross a predefined virtual boundary, a control signal wirelessly transmitted to the power source may alter the current being supplied to a motor. An inertial sensor may provide a movement signal used for generating an association.

### BACKGROUND

In modern surgery, one of the most important instruments available to medical personnel are hand-held surgical tools, such as burs, shavers, drills, saws, wire drivers, ultrasonic tools, and the like. Often these hand-held surgical tools are powered, and therefore it is often desirable for the tool to be cordless for maneuverability and convenience, and therefore powered by a portable power source such as a rechargeable battery.

Navigated-assisted surgery provides for improved presurgical planning and perioperative execution of the presurgical plan. Known systems provide real-time visualization of representations of the instrument relative to patient anatomy through tracking with a localizer detecting a navigation array coupled to the instrument.

Navigated-assisted surgery for hand-held surgical tools is an area of particular interest and development. In certain procedures, for example, a spinal fusion, the vertebral body receives pedicle screws. To do so, it is known to use a drill to create a pilot hole in the cortical wall, a tap to form threads in the pilot hole, and a driver to secure the pedicle screws to the formed threads. Therefore, it may be desirable to have several hand-held surgical tools in the sterile field, yet efficiently transitioning between these instruments in navigated-assisted surgery is fraught with shortcomings. At each instance it is desired to change instruments, the medical personnel may be required interact with a user interface to ensure the system is tracking the correct instrument. For example, the surgeon may have to pause aspects of the procedure, and engage a touchscreen display through several steps for the localizer to detect the navigation array of the instrument desired for use. Systems in which each surgical instrument includes advanced electronics to be independently and simultaneously registered with and controlled by the system are complex and expensive, and the state of the art lacks a navigated-assisted surgical system which provides meaningful integration of legacy instruments with the control-based and other desirable features of navigated-assisted surgery.

### SUMMARY

The navigated-assisted surgical system of the present disclosure overcomes one or more of the aforementioned shortcomings. The navigation-assisted surgery system includes a display unit, a user interface, a memory unit, and a navigation controller in wired or wireless electronic communication with the user interface, the display unit, and the memory unit. A localizer detects and/or senses tracking elements of a navigation array coupled to a hand-held surgical tool the surgical instrument. The navigation controller receives identification signals from the localizer that is tracking the navigation array. The navigation arrays may have differing or unique configurations, arrangements, sizes, and/or shapes of its respective tracking elements. The localizer transmits an identification signal to the navigation controller. The identification signal is indicative of the arrangement of the tracking elements.

The surgical instrument includes a power source. The power source may be a rechargeable battery removably coupled to the hand-held surgical tool. A communication module of the power source is configured to be in wireless communication with a communication module of the navigation-assisted surgery system. The navigation controller may receive at least the battery signal from communication module, and the navigation controller may be further configured to cause transmission of at least a control signal from the communication module to the communication module of the power source.

The associations between the navigation array, the end effector, and the power source may be established in the presurgical planning. The workflow may be carried out on the GUI. The workflow includes the navigation controller receiving the identification signal. The action may occur automatically such that establishing the identity of the navigation array may be accomplished by machine vision. Alternatively, the user may provide a user input to the GUI to establish the identity of the navigation array. The identification signal is provided to the navigation controller, and the identity of the first navigation array may be stored in the memory unit.

The navigation controller may generate a first association between the end effector and the navigation array. A user input to the GUI may include a selection of a type of the end effector. The navigation-assisted surgery system may include a camera configured to capture an image of the end effector, and an algorithm determines the identity the end effector.

The navigation controller receives the battery signal that is wirelessly transmitted from the power source during actuation of the hand-held surgical tool. The power source supplies current to the motor, and the battery signal may be indicative of the current being supplied by or drawn on the power source. The communication module of the power source wirelessly transmits the battery signal to the communication module of the navigation-assisted surgery system. The communication module may transmit the battery signal to the navigation controller. The battery signal or another signal may be indicative of an identity of the power source. The battery signal or the other signal may transmit a code or authentication signature unique to the power source. Alternatively, two-way wireless communication may be established between the power source and the communication module, and the identity of the power source is transmitted to the navigation controller to be stored in the memory unit.

The navigation controller generates a second association between the power source and the navigation array based on the battery signal. The GUI may display a prompt or instructions for the user to actuate the hand-held surgical tool. The prompt may be an arrow or other indicia, for example, an animation, audible feedback, and/or tactical feedback. The navigation controller receives the battery signal and associates the identity of the power source with the identity of the navigation array. It is appreciated that further association between the power source and the end effector, or another identifiable component of the surgical instrument may be provided. The second association is stored in the memory unit. The workflow may be performed any number of additional surgical instruments. The workflow may be repeated for as many surgical instruments as indicated.

The navigated-assisted surgery system independently tracks and controls operation of each of the surgical instruments, particularly through wireless transmission of the control signal to the power source. The navigation controller generates the associations between the navigation array, the end effector, and the power source. Based on the associations, the navigated-assisted surgery system may be configured to, in real-time, determine the position and/or orientation of one or more end effectors, associate it with the predefined virtual boundaries of its respective aspect of the surgical plan, associate it with its power source, maintain two-way wireless communication with the associated power source, and transmit the control signal to the associated power source if the determined the position and/or the orientation of the end effector crosses the associated predefined virtual boundary.

The navigation controller may determine a pose of a patient tracker, and a position and/or an orientation of the end effector based on the pose of the navigation array. The navigation-assisted surgery system is configured to continuously determine the position and/or the orientation of the end effector relative to the predefined virtual boundary. Should the position and/or the orientation of the end effector approach or cross the predefined virtual boundary, the navigation-assisted surgery system is configured to alter operation of the surgical instrument and/or activate an alert device. The navigation-assisted surgery system may control the power source based on the position and/or orientation of the end effector and the pose of the patient tracker. The navigation-assisted surgery system may wirelessly transmit the control signal to the communication module of the power source to change, such as stop, the supply of current or power to the hand-held surgical tool, and more particularly to the motor. The navigation-assisted surgery system may be configured to prevent operation of the surgical instrument when the end effector positioned or angled beyond a threshold magnitude from the aspect of the surgical plan, for example, a target axis. Determining deviations between the end effector and the target depth, target axis, or the like, may be measured relative to a reference location and/or reference coordinate system defined in the known coordinate system.

The navigation-assisted surgery system may be configured to alter operation of the surgical instrument by reducing the current being supplied to the motor. A maximum output power may be reduced to an amount less than that required to operate the surgical instrument at full speed or capacity. The motor may be a variable speed motor and designed to reduce speed when the power required to operate at full speed or capacity is not being received. Reducing the maximum output power of the power source to control maximum speed of the surgical instrument may be related to the identity of the end effector, as certain end effectors are configured for optimal performance at differing speeds and/or torques.

Owing to the stored associations established during the presurgical preparation, the navigation-assisted surgery system may simultaneously control, or at least has the means to control, all of the surgical instruments, thereby allowing the surgeon to handle and move between the surgical instruments without further action. The navigation-assisted surgery system may be agnostic as to which one of the surgical instruments is being handled by the surgeon. Should the predefined virtual boundary be crossed with one of the surgical instruments operating, the navigation may wirelessly transmit the control signal to the appropriate one of the power sources.

It may be desirable for the navigation-assisted surgery system to know which of the surgical instruments is in use. Should the tracking array of only one of the surgical instruments be within the field of view of the localizer, the navigation-assisted surgery system may determine that that surgical instrument is in use. The navigation controller may generate or receive an in-use signal indicative that a particular one of the surgical instruments is in use. The navigation controller may generate the second association between the power source and the navigation array based on the in-use signal. The in-use signal may be stored in the memory unit. In an alternative example in which more than one of the surgical instruments is in the field of view of the localizer, the navigation-assisted surgery system may require the surgeon to provide a “test pull” to determine which one of the surgical instruments is in use. In that moment, the battery signal indicative of the power draw on the power source is wirelessly transmitted from the communication module of the power source to the communication module of the navigation-assisted surgery system. The battery signal is received by the navigation controller, and the navigation controller accesses the stored associations stored in the memory unit. Based on the stored associations, the navigation controller determines which one of the surgical instruments is in use.

The power source may include an inertial sensor configured to generate a movement signal upon handling of the hand-held surgical tool. The inertial sensor is in electronic communication with the communication module of the power source. A movement signal may be generated by the inertial sensor, and wirelessly transmitted to the communication module. The inertial sensor generating the movement signal may also be used during the presurgical preparation to associate the power source, and the navigation array (and/or the end effector). The navigation controller receives the movement signal with the movement signal being indicative of the hand-held surgical instrument being handled or manipulated by the user as detected by the inertial sensor. The navigation controller generates a third association between the power source, and the navigation array based on the movement signal. The third association may be stored in the memory unit.

The GUI may display prompts to induce the user to handle or manipulate one of the surgical instruments. The prompts may be arrows indicative of gestures to be mimicked by the user to generate the movement signal. The movements may be in six degrees of freedom, and the movement signal received by the navigation controller is compared with the gestures displayed on the GUI. Should the movement signals match the gestures, the navigation controller generates the third association based on the movement signal. Any and all functionality of the navigation-assisted surgery system described herein may be realized by the implementation using the inertial sensor. The movement signal may also facilitate the navigation-assisted surgery system determining which surgical instrument is in use.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of an exemplary layout of a surgical environment including a navigated-assisted surgical system, and surgical instruments.

FIG. 2 is a schematic representation of the surgical instruments being within a field of view (FOV) of a localizer of the navigated-assisted surgical system.

FIG. 3 illustrates a display unit displaying a graphical user interface (GUI) showing one step of correlating components of the surgical instrument.

FIG. 4 illustrates the GUI showing another step of correlating the components of the surgical instrument.

FIG. 5 illustrates the GUI showing another step of correlating the components of the surgical instrument.

FIG. 6 is a schematic view of a surgical instrument positioned and oriented relative to predefined virtual boundaries and predefined virtual zones. Certain components of the navigated-assisted surgical system and the surgical instrument are represented schematically.

FIG. 7 is representation of a user capable of manipulating the surgical instrument in six degrees of freedom so as to mimic gestures being displayed on the GUI for correlating the components of the surgical instrument.

### DETAILED DESCRIPTION

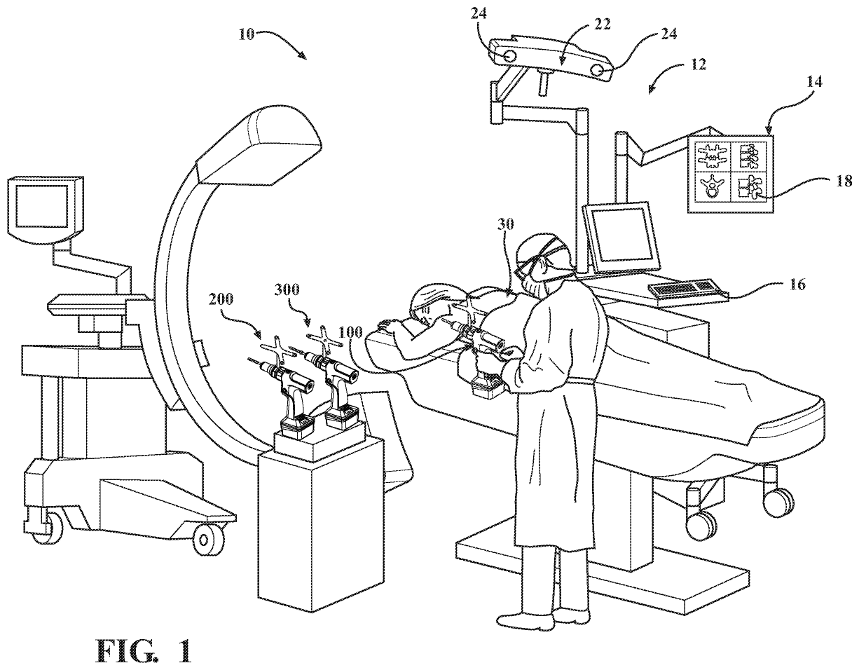

FIG. 1 illustrates a surgical environment 10 including a navigation-assisted surgery system 12 for correlating components of at least one surgical instrument 100, 200, 300 to be used in navigation-assisted surgery. The association of the components may be performed during presurgical preparations by an operating room technician or other medical personnel, or prior to a surgeon executing a surgical plan of a medical procedure. The navigation-assisted surgery system 12 may further track and control the surgical instruments 100, 200, 300 to assist the surgeon in executing the medical procedure.

The navigation-assisted surgery system 12 includes a display unit 14, and a user interface 16. The display unit 14 and the user interface 16 may be integrated on a touchscreen display for providing a graphical user interface (GUI) 18. The display unit 14 is configured to display information such as preoperative patient imaging and the surgical plan. For example, the surgical plan may include a predefined trajectory for an implant such as a pedicle screw. The surgical plan may also include overlaying the position and/or orientation of the implant on the patient imaging or patient data. The GUI 18 may be configured to allow the surgeon to input, select, edit, and/or manipulate the patient data and/or the surgical plan. In the spinal fusion procedure, the surgeon may enter information related to position, orientation, target location, and target depth in which the pedicle screws are to be implanted, and/or the size and shape of the pedicle screw. The surgeon may also be able to identify critical anatomical features such cortical walls, nerves, blood vessels or similar critical anatomical structures, from which predefined virtual boundaries or zones may be utilized by the navigation-assisted surgery system 12 to facilitate execution of the surgical plan.

The navigation-assisted surgery system 12 includes a navigation controller 20. The navigation controller 20 is in wired or wireless electronic communication with the user interface 16, the display unit 14, central processing unit (CPU) and/or other processors, a memory unit 26, and other hardware. The navigation controller 20 may include non-transitory computer memory for executing software and/or operating instructions related to the operation of the navigation-assisted surgery system 12 and to implement the methods disclosed herein. A localizer 22 includes one or more sensors 24 adapted to detect and/or sense the position of tracking elements 135, 235, 335 of a navigation array 130, 230, 330 coupled to a hand-held surgical tool 120, 220, 320 the surgical instrument 100, 200, 300. One suitable localizer is disclosed in commonly-owned U.S. Pat. No. 10,531,926, issued Jan. 14, 2020, the entire contents of which is hereby incorporated by reference. The localizer 22 is in communication with the navigation controller 20, and the navigation controller 20 is configured to receive identification signals from the localizer 22 that is tracking the navigation array 130, 230, 330.

In the illustrated implementation, each of the hand-held surgical tools 120, 220, 320 may have the similar or same architecture in which a number of similar components are capable of performing similar functions and/or operations to be described. In alternative implementations, the hand-held surgical tools 125, 225, 325 may have a different construction. The hand-held surgical tool 125, 225, 325 shaped to define a handle or grip portion for the surgeon to hold while performing the medical procedure. The hand-held surgical tool 125, 225, 325 may include a motor 145, 245, 345, and the surgical instrument 100, 200, 300 includes an end effector 140, 240, 340 configured to be operably coupled to the motor 145, 245, 345. In the example of spinal fusion, the first end effector 140 may be a drill bit such that the first surgical instrument 100 operates as a drill, the second end effector 240 may be a tap bit such that the second surgical instrument 200 operates as a tap, and the third end effector 340 may be a driver tip configured to engage the pedicle screw such that the third surgical instrument 300 operates as a driver. The motor 145, 245, 345 may be configured to rotate the end effector 140, 240, 340 to bore the pilot hole, tap the pilot hole, and/or drive the pedicle screw. Other surgical instruments contemplated to be used with the navigation-assisted surgery system 12 of the present disclosure includes an ultrasonic cutting instrument, a bur, a shaver, a bone saw, and an ablation electrode, among others.

The navigation array 130, 230, 330 may be removably coupled to the hand-held surgical tool 125, 225, 325 (and/or the end effector 140, 240, 340) of the surgical instrument 100, 200, 300. One suitable coupler for removably coupling the navigation array 130, 230, 330 to the hand-held surgical tool 125, 225, 325 is disclosed in commonly-owned U.S. Provisional Patent Application No. 63/154,273 filed Feb. 26, 2021, the entire contents of which are hereby incorporated by reference. The tracking elements 135, 235, 335 of the navigation array 130, 230, 330 may be passive tracking elements (e.g., reflectors) for reflecting light emitted from the localizer 22, active tracking elements (e.g., light emitting diodes), or a combination thereof. Alternatively, the navigation-assisted surgery system 12 may utilize electromagnetic, radiofrequency, or other suitable means for tracking a position and orientation (pose) of the navigation array 130, 230, 330. Calibration data associated with the end effector 140, 240, 340 stored in the memory unit 26 is associated with a respective one of the navigation arrays 130, 230, 330 in manners to be described, after which the navigation controller 20 can determine a position and/or orientation of the end effector 140, 240, 340, for example, a tip of the end effector 140, 240, 340.

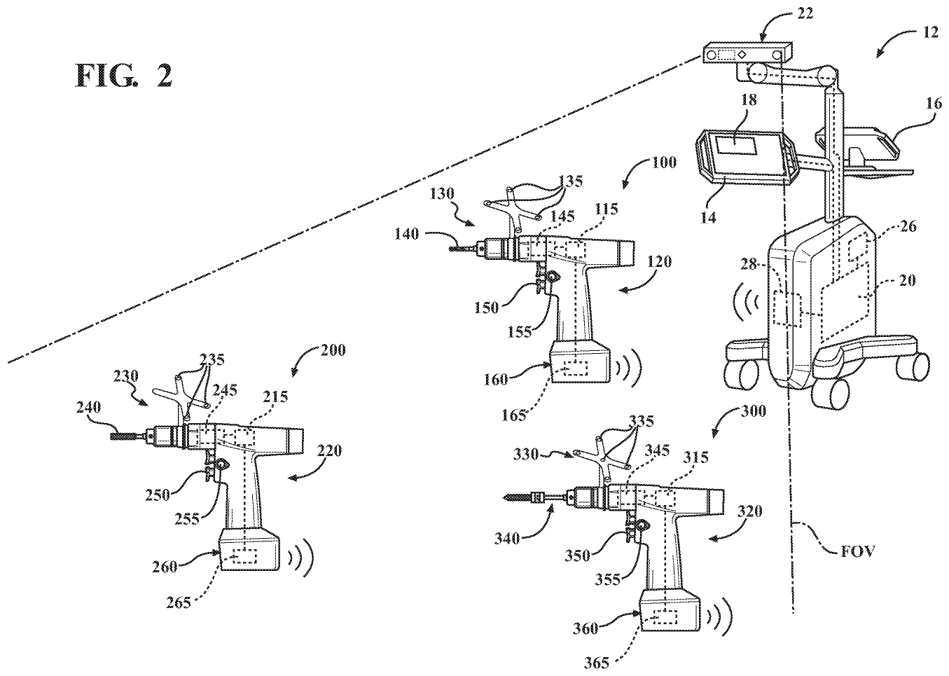

The navigation arrays 130, 230, 330 may have differing or unique configurations, arrangements, sizes, and/or shapes of its respective tracking elements 135, 235, 335 such that the localizer 22 is configured to distinguish between the first navigation array 130, the second navigation array 230, and the third navigation array 330. The differing arrangements are schematically represented by the unique geometries shown in FIGS. 1 and 2. More particularly, based on the arrangement and/or spatial orientation of the tracking elements 135, 235, 335, the localizer 22 transmits the identification signal to the navigation controller 20, which is used for generating certain associations between components of the surgical instrument 100, 200, 300 to be further described. In alternative configurations, such as configurations where the tracking elements 135, 235, 335 are configured to pulse in a manner specific to the corresponding navigation array 130, 230, 330, the localizer 22 may transmit the identification signal to the navigation controller 20 based on detecting a pulse pattern of the tracking elements 135, 235, 335 of the navigation arrays 130, 230, 330. In still other configurations, each array may include its own unique identifier, and may transmit the unique identifier to the localizer, using a transmitter, such as an antenna or infrared sensor/emitter. In another alternative configuration, such as a configuration where the tracking elements 135, 235, 335 are configured to transmit a navigation array signal specific to the corresponding navigation array 130, 230, 330 (e.g. configurations where the tracking elements 135, 235, 335 are antennas), the localizer 22 may transmit the identification signal to the navigation controller 20 based on receiving a navigation array signal from the tracking elements 135, 235, 335 of the navigation arrays 130, 230, 330.

With continued reference to FIG. 2, the surgical instrument 100, 200, 300 includes a power source 160, 260, 360, for example, a rechargeable battery. The power source 160, 260, 360 is configured to be removably coupled with the hand-held surgical tool 120, 220, 320 to be in electrical communication with the motor 145, 245, 345. The power source 160, 260, 360 is configured to selectively provide power to the motor 145, 245, 345 to operate the end effector 140, 240, 340, for example, upon actuation of a switch 150, 250, 350 of the hand-held surgical tool 120, 220, 320. Energizing the motor 145, 245, 345 results in a change in a characteristic of energy being supplied by the power source 160, 260, 360. The characteristic of energy may be a current draw or a voltage drop in the power source 160, 260, 360 corresponding to the current supplied to the motor 145, 245, 345. A battery signal may be indicative of the current draw or voltage drop of the power source 160, 260, 360. In certain implementations, the first surgical instrument 100 includes a first power source 160, the second surgical instrument 200 includes a second power source 260, and the third surgical instrument 300 includes a third power source 360. However, it is contemplated that the surgical environment 10 may include fewer power sources than hand-held surgical tools, and the power source(s) may be interchangeably moved between and correlated with the hand-held surgical tools in a manner to be further described.

The power source 160, 260, 360 includes a communication module 165, 265, 365. The communication module 165, 265, 365 of the power source 160, 260, 360 is configured to be in wireless communication with a communication module 28 of the navigation-assisted surgery system 12. One exemplary wireless technology to facilitate the wireless communication between the communication modules 28, 165, 265, 365 is Bluetooth® low energy protocol, however, Wi-Fi, cellular, and other wireless technologies may be utilized. The communication module 165, 265, 365 of the power source 160, 260, 360 may be a transceiver configured to send signals to and receive signals from the communication module 28 in electronic communication with navigation controller 20. More particularly, the navigation controller 20 may be configured to receive at least the battery signal from communication module 28, and the navigation controller 20 may be further configured to cause transmission of at least a control signal from the communication module 28 to the communication module 165, 265, 365 of the power source 160, 260, 360. Additional data and/or signals may be exchanged, for example, signals indicative of a battery level, alarms or alerts, or the like.

For convenience and efficiency in executing the surgical plan, it may be desirable for several hand-held surgical tools to be near the surgical field, and more particularly concurrently within a field of view of the localizer 22. FIG. 1 shows the surgeon holding the first surgical instrument 100, and the second and third surgical instruments 200, 300 positioned in a proximity to the surgical site 30 so as to be detectable by the localizer 22. FIG. 2 schematically represents the first, second, and third surgical instruments 100, 200, 300 being in the field of view (FOV) of the localizer 22. The efficiency is realized with the navigated-assisted surgery system 12 of the present disclosure being configured to independently track and control operation of each of the surgical instruments 100 wirelessly, particularly through wireless transmission of the control signal to the power source 160, 260, 360. By leveraging the power source 160, 260, 360 to do so, the internal hardware of the hand-held surgical tool 120, 220, 320 need not necessarily include advanced electronics associated with added complexity and cost of manufacture and assembly. Further, doing so may provide for tracking and control of legacy surgical instruments, thereby not requiring replacing a suite of surgical tools in order to retrofit the instruments with navigated-assisted capabilities.

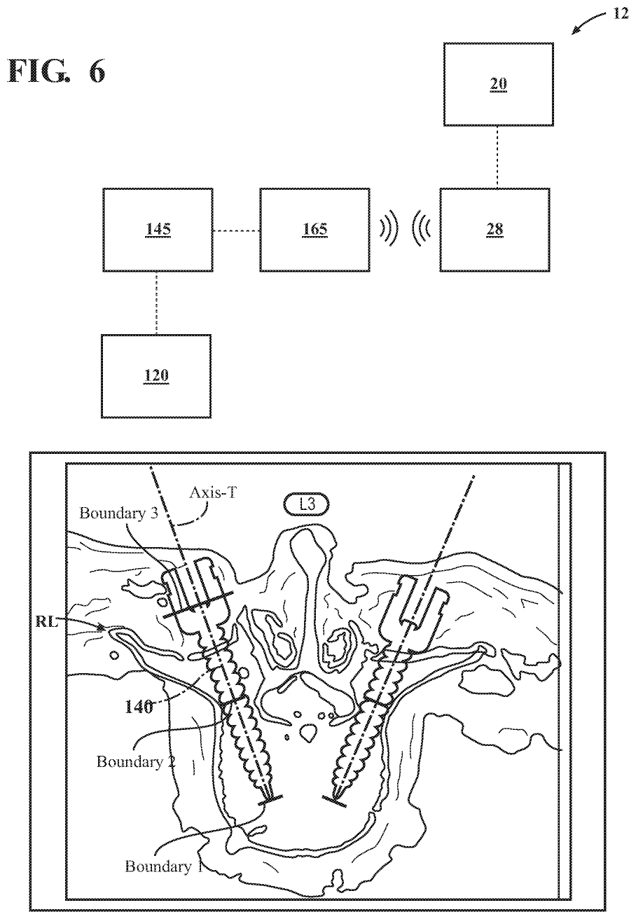

To achieve such advantageous functionality, associations between the navigation array 130, 230, 330, the end effector 140, 240, 340, and/or the power source 160, 260, 360 are generated or determined by the navigation controller 20 of the navigated-assisted surgery system 12, and stored in the memory unit 26. Stated differently, based on signals received during presurgical preparation and/or during execution of the procedure, the navigation controller 20 may determine which of multiple end effectors and multiple power sources is coupled to a given navigation array. For example, the navigation controller 20 may determine the drill bit and the first power source 160 are associated with the first navigation array 130, the tap bit and the second power source 260 are associated with the second navigation array 230, and the driver tip and the third power source 360 are associated with the third navigation array 330. The aforementioned associations account for the end effectors 140, 240, 340 being correlated to different aspects of the surgical plan in which differing predefined virtual boundaries or zones are defined based on the end effector 140, 240, 340 itself. In other words, for example, a depth to which the pedicle screw may be permitted to be driven into the vertebra may be greater than a depth to which the drill is permitted to prepare the pilot hole. Certain aspects of controlling the surgical instrument 100, 200, 300 will be further described with reference to FIG. 6, and additional disclosure is further described in commonly-owned International Application No. PCT/US2020/053092, filed Sep. 28, 2020, the entire contents of which is hereby incorporated by reference. In short, the navigated-assisted surgery system 12 is configured to, in real-time, determine the position and/or orientation of one or more end effectors 140, 240, 340 relative to the predefined virtual boundaries of its respective aspect of the surgical plan; and by maintaining two-way wireless communication with the associated power source 160, 260, 360, and the control signal may be transmitted to the associated power source 160, 260, 360 if the determined position and/or the orientation of the end effector 140, 240, 340 crosses the associated predefined virtual boundary.

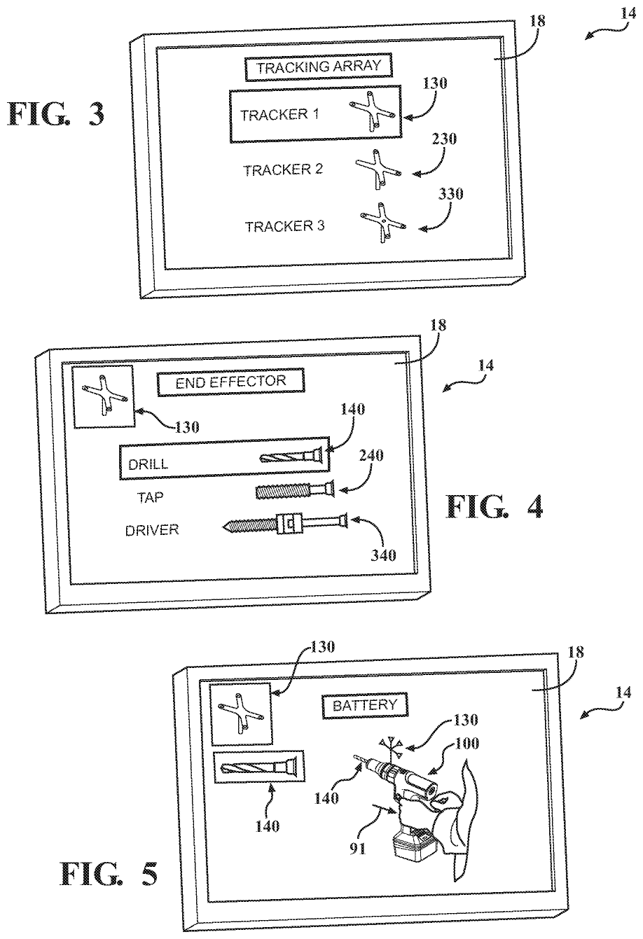

The associations between the navigation array 130, 230, 330, the end effector 140, 240, 340, and the power source 160, 260, 360 may be established in the presurgical planning. It is also appreciated that, for any number of reasons, it may be indicated to establish or reestablish the associations during execution of the surgical plan. Referring now to FIGS. 3-5, an exemplary workflow of establishing the correlations is described. The exemplary workflow to be described may be considered “navigation array centric” in which the identity of the navigation array 130, 230, 330 is initially established, and the end effector 140, 240, 340 and the power source 160, 260, 360 are associated to the identity of the navigation array 130, 230, 330. However, it is contemplated that alternative methods may be “end effector centric” or “power source centric” in which the identity of the end effector 140, 240, 340 or the power source 160, 260, 360, respectively, is initially established, and the other components are associated to the identity of the same. Further methods to be described include automatic detection of the identity of one or more of the navigation arrays 130, 230, 330, the end effectors 140, 240, 340, and the power sources 160, 260, 360 using machine vision or some alternative data reader, such as an optical reader or a radiofrequency identification reader.

The workflow may be carried out on the GUI 18 being a touchscreen display of the display unit 14. The workflow includes the navigation controller 20 receiving the identification signal indicative of the navigation array 130, 230, 330. After the workflow is initiated, the GUI 18 may display a prompt for the user to move the first surgical instrument 100 to which the first navigation array 130 is coupled in the field of view of the localizer 22. The localizer 22 detects the arrangement of the first tracking elements 135 of the first navigation array 130, and based on the arrangement, provides the identification signal to the navigation controller 20. The navigation controller 20 may compare the identification signal to calibration data from a database of predefined navigation arrays. Should the identification signal match the calibration data of the first navigation array 130, the navigation controller 20 stores the identity of the first navigation array 130 in the memory unit 26. The aforementioned actions may occur automatically such that establishing the identity of the navigation array 130, 230, 330 may be accomplished by machine vision. As shown in FIG. 3, the GUI 18 may provide visual emphasis to alert the user that the first navigation array 130 has been successfully identified, and a pictorial representation of the first navigation array 130 may be provided for the user to perform a visual confirmation if desired.

In certain implementations, the user may provide an input to the GUI 18 to establish the identity of the navigation array 130, 230, 330 after being prompted to do so. The GUI 18 may provide a pictorial representation of all of the navigation arrays 130, 230, 330 being detected by the localizer 22 at that time. The user may then select on the GUI 18 which one of the navigation arrays 130, 230, 330 to continue with further setup. FIG. 3 may represent such example in which the localizer 22 has detected the first, second, and third navigation arrays 130, 230, 330, and the GUI 18 is providing pictorial representations of the same (in addition to text-based outputs). The user may select, on the GUI 18, the navigation array 130, 230, 330 based on the arrangement of the tracking elements 135, 235, 335 matching that of the tracking elements 135, 235, 335 supported by the hand-held surgical tool 120, 220, 320 being held by the user at that moment. In other words, the user may hold one of the hand-held surgical tools 120, 220, 320, observe the arrangement of the tracking elements 135, 235, 335, and select on the GUI 18 the navigation array 130, 230, 330 matching the same. In the illustrated example, the user selects first navigation array 130, after which its icon may be visually emphasized. The identification signal is provided to the navigation controller 20, and the identity of the first navigation array 130 may be stored in the memory unit 26.

Referring to FIG. 4, on a subsequent output of the GUI 18, the workflow may include the step of generating, with the navigation controller 20, a first association between the end effector 140, 240, 340 and the navigation array 130, 230, 330. The input may be a user input to the GUI 18 that includes a selection of a type of the end effector 140, 240, 340. In a previous step in the workflow, a type of the surgical procedure may be inputted, and the GUI 18 may display one or more of the end effectors 140, 240, 340 indicated for the type of surgical procedure. The end effectors 140, 240, 340 indicated for the spinal fusion procedure, for example, may include the drill bit, the tap bit, and the driver tip, as shown in FIG. 4. Alternatively, a list of all potential end effectors may be provided on the GUI 18, for example, in a scrollable list. The user may select, on the GUI 18, the end effector 140, 240, 340 matching that of the end effector 140, 240, 340 coupled to the hand-held surgical tool 120, 220, 320 being held by the user at that moment. In other words, the user may hold one of the hand-held surgical tools 120, 220, 320, observe the end effector 140, 240, 340, and select the appropriate end effector 140, 240, 340 on the GUI 18. In the illustrated example, the user selects first end effector 140, after which its icon may be visually emphasized. The input is provided to the navigation controller 20, and the input may be stored the memory unit 26.

In an alternative implementation, the navigation-assisted surgery system 12 include a camera (not shown) configured to capture an image of the end effector 140, 240, 340. The image is processed by an algorithm in which the characteristics of the end effector 140, 240, 340 identifiable in the captured image is used to automatically determine the identity of the end effector. The input provided to the navigation controller 20 is generated by machine vision.

The GUI 18 may display the pictorial representation of the identity of the navigation array 130, 230, 330 previously established during the step of receiving the input indicative of the identity of the end effector 140, 240, 340. FIG. 4 shows the first navigation array 130 being displayed based on GUI 18, based on the selection from FIG. 3, for the convenient reference of the user during subsequent steps of the workflow.

The navigation controller 20 is configured to generate the first association between the end effector 140, 240, 340 and the navigation array 130, 230, 330 based on the input and the identification signal. The first association may include associating the calibration data of the end effector 140, 240, 340 with the navigation array 130, 230, 330, and more particularly the tracking elements 135, 235, 335 of the navigation array 130, 230, 330. As a result, the navigation controller 20 may determine and track the position and/or orientation of the end effector 140, 240, 340 so long as the tracking elements 135, 235, 335 remain within the field of view of the localizer 22. The first association may be stored in the memory unit 26.

Referring now to FIG. 5, the workflow further includes receiving with the navigation controller 20 the battery signal that is wirelessly transmitted from the power source during actuation of the hand-held surgical tool 120, 220, 320. More particularly, during actuation of the switch 150, 250, 350 or other electrical component capable of controlling the energy supplied by power source 160, 260, 360 to the instrument, the power source 160, 260, 360 supplies current to the motor 145, 245, 345, and the battery signal may be indicative of the current being supplied by or drawn on the power source 160, 260, 360. The communication module 165, 265, 365 of the power source 160, 260, 360 wirelessly transmits the battery signal to the communication module 28 of the navigation-assisted surgery system 12. The communication module 28 may transmit the battery signal to the navigation controller 20.

The battery signal or another signal may be indicative of an identity of the power source 160, 260, 360. In other words, the battery signal or the other signal may transmit a code or authentication signature unique to the power source 160, 260, 360. Alternatively, during a previous step in the workflow, two-way wireless communication may be established between the power source 160, 260, 360 and the communication module 28, and the identity of the power source 160, 260, 360 is transmitted to the navigation controller 40 and stored in the memory unit 26. For example, the power source 160, 260, 360 may be “paired” to establish the Bluetooth® wireless communication, and thereafter the communication module 28 may instantaneously and/or simultaneously send signals to one or more of the power sources 160, 260, 360 through known means of the Bluetooth communication protocol.

The navigation controller 20 generates a second association between the power source 160, 260, 360 and the navigation array 130, 230, 330 based on the battery signal during actuation of the hand-held surgical tool 120, 220, 320. Because the setup pertains to a known one of the navigation arrays 130, 230, 330 and because the navigation array 130, 230, 330 is in the field of view of the localizer 22, when the hand-held surgical tool 120, 220, 320 is actuated, the navigation controller 20 receives the battery signal and associates the identity of the power source 160, 260, 360 with the identity of the navigation array 130, 230, 330. It is appreciated that the second association or another association may be further between the power source 160, 260, 360 and the end effector 140, 240, 340, or another identifiable component of the surgical instrument 100, 200, 300. The second association is stored in the memory unit 26, for example, the association may be stored in a look-up table.

To facilitate the generation of the second association, the GUI 18 may display a prompt 91 or instructions for the user to actuate the hand-held surgical tool 120, 220, 320. FIG. 5 shows the pictorial representation of the surgical instrument 100, 200, 300. The exemplary prompt 91 is an arrow near a position of the switch 150, 250, 350 to instruct the user to engage the switch 150, 250, 350 (e.g., pull a trigger). Other types of prompts are contemplated, for example, an animation or other visual indicia, audible feedback, and/or tactical feedback that provides intuitive instruction to the user to complete the present step of the workflow. The GUI 18 may also display the pictorial representation of the identity of the navigation array 130, 230, 330 and/or the identity of the end effector 140, 240, 340 previously established. FIG. 5 shows the first navigation array 130 and the first end effector 140 being displayed based on GUI 18, based on the selection/inputs from FIGS. 3 and 4, for the convenient reference of the user. Further, the pictorial representation may be rendered with representations of the navigation array 130, 230, 330 and/or the end effector 140, 240, 340 previously established. FIG. 5 shows the first navigation array 130 and the first end effector 140 coupled to the first hand-held surgical tool 120, and its appearance should match that of the first surgical instrument 100 being held by the user. For example, the GUI 18 may illustrate one or more colors or alphanumeric characteristics associated with the tracking array 130, 230, 330 that was associated with the identity of the end effector 140, 240, 340.

The workflow previously described may be performed for a single one of the surgical instruments 100, 200, 300. In exemplary implementations, the same or similar preoperative setup may be carried out for the second instrument 200, the third surgical instrument 300, and any number of additional surgical instruments. Subsequent to the surgical instrument(s) being registered by the correlating of its components with the navigation-assisted surgery system 12, the surgical procedure may proceed with execution of the surgical plan. The surgical plan may be stored in the memory unit 26. Preoperatively, the anatomical model is registered to a patient tracker (not shown) coupled to the patient such that the virtual boundaries become associated with the anatomical model and an associated coordinate system. The patient tracker may be, for example, a navigation array rigidly secured to the vertebra or other bone of the patient. The patient tracker including tracking elements detectable by the localizer 22, and therefore the pose of the patient tracker may be determined by the navigation controller 20. The tracking elements 135, 235, 335 of the navigation array 130, 230, 330 are also detectable by the localizer 22, and a pose of the navigation array 130, 230, 330 may be determined by the navigation controller 20. The navigation controller 20 may further determine a position and/or an orientation of the end effector 140, 240, 340 based on the pose of the navigation array 130, 230, 330.

Referring now to FIG. 6, the navigation-assisted surgery system 12 may be configured to define an alert zone and/or boundary corresponding to a target depth or a maximum depth for each of the end effectors 140, 240, 340. For example, a first virtual boundary representing target depth for a drill to bore the hole, a second virtual boundary representing target depth for the tap, and a third virtual boundary representing target depth for the driver to insert the screw. These multiple virtual boundaries may be activated, one at a time, by the navigation controller 20.

FIG. 6 shows a schematic representation of the predefined virtual boundaries (Boundary 1, 2, 3) overlaying the patient imaging of the patient anatomy of the surgical site 30. The virtual boundaries may be used in various ways. For example, the navigation processor 140 may/or control certain operations/functions of the hand-held surgical instruments 120, 220, 320 based on a relationship of the hand-held surgical instruments 120, 220, 320 and/or the associated end effectors to the boundary (e.g., spatial, velocity, etc.). Other uses of the boundaries are also contemplated.

Boundaries to ensure that instruments are positioned at the desired depth may be defined by a virtual planar boundary, a virtual volumetric boundary, or other forms of virtual boundary. Virtual boundaries may also be referred to as virtual objects. The virtual boundaries may be defined with respect to an anatomical model, such as a 3D bone model. In other words, the points, lines, axes, trajectories, planes, volumes, and the like, that are associated with the virtual boundaries may be defined in a coordinate system that is fixed relative to a coordinate system of the anatomical model such that tracking of the anatomical model (e.g., via tracking the associated anatomy to which it is registered) also enables tracking of the virtual boundary.

In instances where the navigation-assisted surgery system 12 includes more than one surgical instrument 100, 200, 300, the navigation controller 20 may be optionally configured to select a predefined virtual boundary based on an end effector 140, 240, 340 associated with an in-use navigation array 130, 230, 330. For example, in instances where the navigation-assisted surgery system 12 includes the first surgical instrument 100 including the first end effector 140 and the second surgical instrument 200 including the second end effector 240 of a different type than the first end effector 140, the navigation controller 20 may determine whether the navigation array 130 associated with the first end effector 140 or the second navigation array 230 associated with the second end effector 240 is in use. The navigation controller 20 may then select the predefined virtual boundary based on the end effector corresponding to the in-use navigation array. The method and system by which the navigation controller 20 determines whether a navigation array 130, 230, 330 is in use will be described in greater detail below. By providing this check of whether the navigation array is in-use, the system can help mitigate the risk of inadvertently controlling the wrong surgical instrument based on the intended virtual boundary. In certain implementations of the system, the act of checking whether the array is in-use is omitted. In such an implementation, the first and second associations may be used instead.

The virtual boundaries may be one-dimensional, two-dimensional, or three-dimensional, and may include a volume or other shapes, including complex geometric shapes. The virtual boundaries may be represented by pixels, point clouds, voxels, triangulated meshes, and the like. The virtual boundaries may be implant-specific (e.g., defined based on a size and shape, of the pedicle screw) and/or patient-specific (e.g., defined based on the patient's anatomy). The virtual boundaries may be boundaries that are created preoperatively, intraoperatively, or combinations thereof. The virtual boundaries may be defined in a coordinate system that is fixed relative to a coordinate system of the anatomical model such that tracking of the anatomical model also enables tracking of the virtual boundary. The virtual boundaries may be stored in the memory unit 26 for retrieval and/or updating. Further disclosure regarding defining the boundaries and alert zones is disclosed in the aforementioned International Patent Application No. PCT/US2020/053092, and further disclosed in commonly-owned U.S. Pat. No. 7,747,311, issued Jun. 29, 2010, U.S. Pat. No. 8,898,043, issued Nov. 25, 2014, and United States Patent Publication No. 2018/0333207, published Nov. 22, 2018, the entire contents of each are hereby incorporated by reference.

The navigation-assisted surgery system 12 may be configured to continuously determine the position and/or the orientation of the end effector 140, 240, 340 relative to the virtual boundary or boundaries. The navigation-assisted surgery system 12 may do so simultaneously for all of the surgical instruments 100, 200, 300 within the field of view of the localizer 22. Should the end effector 140, 240, 340 be away from the virtual boundary, the surgical instruments 100, 200, 300 may be operated normally. Should the position and/or the orientation of the end effector 140, 240, 340 approach or cross a predefined virtual boundary, the navigation-assisted surgery system 12 is configured to alter operation of the surgical instrument 100, 200, 300 and/or activate an alert device 255, 355, 455. One exemplary manner in which the navigation-assisted surgery system 12 alters operation of the surgical instrument 100, 200, 300 is at least temporarily preventing its operation. To do so, the navigation-assisted surgery system 12 may control and/or communicate with the power source 160, 260, 360 based on the position and/or orientation of the end effector 140, 240, 340 and the pose of the patient tracker or pose of the patient determined in an alternative method, such as using a surface topology system. In an exemplary implementation, the communication module 28 of the navigation-assisted surgery system 12 may wirelessly transmit the control signal to the communication module 165, 265, 365 of the power source 160, 260, 360. The control signal may be to change, such as stop, the supply of current or power to the hand-held surgical tool 120, 220, 320, and more particularly to the motor 145, 245, 345. With no available power, the motor 145, 245, 345 is deactivated, and the surgical instrument 100, 200, 300 is prevented from operation. The control signal may alternatively be to brake the motor 145, 245, 345. The motor 145, 245, 345 and drive train of the hand-held surgical tool 120, 220, 320 may be designed to limit any latency from power cessation to the end effector 140, 240, 340 becoming static.

For example, and with continued reference to FIG. 6, the first end effector 140 is at pose relative to the target depth (T) on the target axis (Axis-T). The first end effector 140 is the drill bit configured to bore a pilot hole through the pedicle of the vertebra (the surgical site 30) along the target axis to the target depth that is defined in the surgical plan. The target axis, target depth, or other aspect of the surgical plan may have been defined by the surgeon and/or automatically generated based on segmentation of the patient image data and an algorithm that determines the optimal target axis for a given patient anatomy. Should the first end effector 140 approach or cross Boundary 1, the navigation controller 20 wirelessly transmits the control signal to the first power source 160 to alter, such as terminate, current being supplied to the first motor 145 or to brake the first motor 145. The cessation of operation of the first surgical instrument 100 should be discernable to the surgeon.

It is also contemplated that the navigation-assisted surgery system 12 may be configured to prevent operation of the surgical instrument 100, 200, 300 when the end effector 140, 240, 340 positioned or angled beyond a threshold magnitude from the surgical plan. As opposed to requiring the predefined virtual boundary to be offended, the surgical instrument 100, 200, 300 may not be operable unless within the threshold magnitude from the patient anatomy of interest. Determining deviations between the end effector 140, 240, 340 and the target depth, or the like, may be measured relative to a reference location (RL) and/or reference coordinate system defined in the known coordinate system.

In certain implementations, the navigation-assisted surgery system 12 may be configured to alter operation of the surgical instrument 100, 200, 300 by reducing the current being supplied to the motor 145, 245, 345. Through appropriate circuitry of the power source 160, 260, 360, a maximum output power may be reduced to an amount less than that required to operate the surgical instrument 100, 200, 300 at full speed or capacity. The motor 145, 245, 345 may be a variable speed motor and correspondingly designed to reduce speed when the power required to operate at full speed or capacity is not being received. Such an arrangement may be particularly well suited for situations where the end effector 140, 240, 340 is approaching but has not crossed the predefined virtual boundary. The gradual slowing of the surgical instrument 100, 200, 300 may be less abrupt from the perspective of the surgeon, and preventing operation of the surgical instrument 100, 200, 300 should the surgeon continue towards and cross the predefined virtual boundary may be used in combination.

Furthermore, reducing the maximum output power of the power source 160, 260, 360 to control maximum speed of the surgical instrument 100, 200, 300 may be related to the identity of the end effector 140, 240, 340. Certain end effectors 140, 240, 340 are configured for optimal performance at a defined speed and/or torque. For example, the first surgical instrument 100 being a drill may operate in a high speed and low torque mode, whereas the third surgical instrument 300 being the driver may operate in a low speed and high torque mode. Owing to the first and second associations, namely the associations between the end effector 140, 240, 340 and the power source 160, 260, 360, respectively, with the navigation array 130, 230, 330, the identity of the end effector 140, 240, 340 and the power source 160, 260, 360 can be controlled accordingly.

In addition to controlling operation of the surgical instrument 100, 200, 300, the navigation-assisted surgery system 12 of the present disclosure advantageously provides for improved transitioning between the surgical instruments 100, 200, 300 during execution of the surgical plan. As mentioned, it may be desirable for multiple surgical instruments 100, 200, 300 to be placed in the operating room in a convenient location for the surgeon relative to the surgical site 30. Yet, known systems may require the surgeon or other user to pause an aspect of the surgical procedure to engage the user interface to inform the system that a change to the surgical instrument is being made. Owing to the stored associations established during the presurgical preparation, the navigation-assisted surgery system 12 may simultaneously control, or at least has the means to control, all of the surgical instruments 100, 200, 300, thereby allowing the surgeon to handle and move between the surgical instruments 100, 200, 300 without further action or input to the GUI 18. In other words, the navigation-assisted surgery system 12 may be agnostic as to which one of the surgical instruments 100, 200, 300 is being handled by the surgeon. Rather, the navigation-assisted surgery system 12 may be only concerned with the positions and/or orientations of the end effectors 140, 240, 340 relative the predefined virtual boundaries registered to the patient tracker. Should the predefined virtual boundary be crossed with one of the surgical instruments 100, 200, 300 operating, the navigation controller 20 is receiving the battery signal from the power source 160, 260, 360 (from the current draw or voltage drop) or identification signal and therefore may wirelessly transmit the control signal to the appropriate one of the power sources 160, 260, 360.

In certain implementations, it may be desirable for the navigation-assisted surgery system 12 to know which of the surgical instruments 100, 200, 300 is in use. “In use” may be considered to mean the hand-held surgical tool 120, 220, 320 being handled by the surgeon to be applied to the surgical site 30.

The navigation controller 20 may generate or receive an in-use signal indicative that a particular one of the surgical instruments 100, 200, 300. The navigation controller 20 may generate the second association between the power source 160, 260, 360 and the navigation array 130, 230, 330 (and/or the end effector 140, 240, 340) based on the in-use signal. The in-use signal may be stored in the memory unit 26.

In an alternative example in which more than one of the surgical instruments 100, 200, 300 may be in the field of view of the localizer 22, the navigation-assisted surgery system 12 may require the surgeon merely provide a “test pull” to the switch 150, 250, 350 of the hand-held surgical tool 120, 220, 320 to determine which one of the surgical instruments 100, 200, 300 is in use.

It is also contemplated that the aforementioned “test pull” may be utilized during the preoperative preparation to generate the associations. The workflow may include the surgeon momentarily operating the surgical instrument 100, 200, 300 in the field of view of the localizer 22. In that moment, the battery signal indicative of the power draw on the power source 160, 260, 360 is wirelessly transmitted from the communication module 165, 265, 365 of the power source 160, 260, 360 to the communication module 28 of the navigation-assisted surgery system 12. The battery signal is received by the navigation controller 20, and the navigation controller 20 accesses the stored associations stored in the memory unit 26. Based on the stored associations, the navigation controller 20 determines which one of the surgical instruments 100, 200, 300 is in use and instantaneously determines its position and/or orientation relative to the predefined virtual boundary or target axis. The navigation controller 20 determines which one or more of the first surgical instrument 100, the second surgical instrument 200, and third surgical instrument 300 is in use. The navigation-assisted surgery system 12 may still control operation of all of the surgical instruments 100, 200, 300 simultaneously as described, but one of the surgical instruments 100, 200, 300 may be considered in use for realizing additional functionality of navigation-assisted surgery system 12 to be described.

In particular, the navigation controller 20 may be configured to refresh or update the display unit 14 to show the aspect of the surgical plan relevant to which surgical instrument 100, 200, 300 is in use. For example, upon preparing the vertebrae, the surgeon may handle the third surgical instrument 300 that is a driver, and actuate the “test pull.” The aforementioned actions occur such that the navigation controller 20 accesses from the memory unit 26 the aspect of the surgeon plan associated with inserting the pedicle screw. The navigation controller 20 may transmit a display signal to the display unit 14 to automatically update the GUI 18. The update may include providing a digital representation of the pedicle screw on the GUI, or any other desired visual change. Further, the surgeon's display preferences, perhaps for each aspect of the surgical plan, may be defined preoperatively; and the display preferences may be automatically updated as the surgeon moves between the aspects of the surgical plan. Preferences may include anatomical views (e.g., sagittal, axial, etc.), arrangement of the anatomical views, on-screen indicia, and/or any other visual preference for the GUI 18 that may be inputted and saved in software.

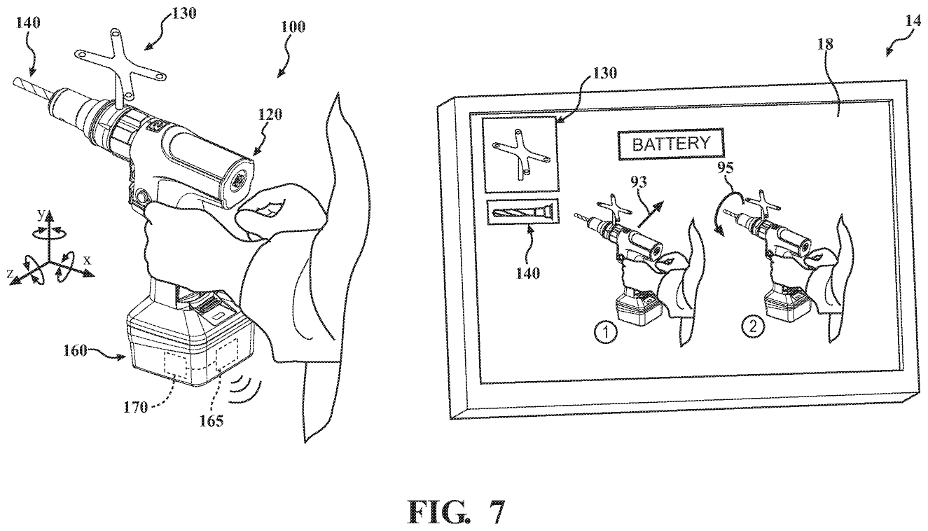

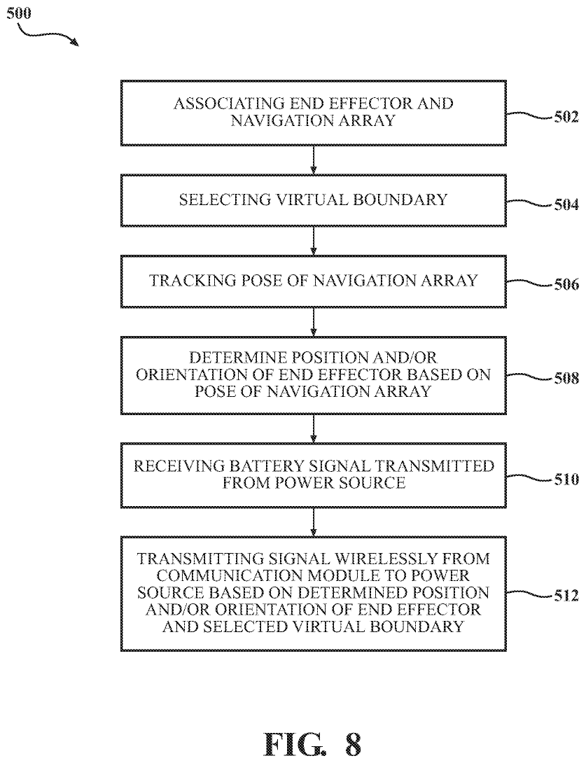

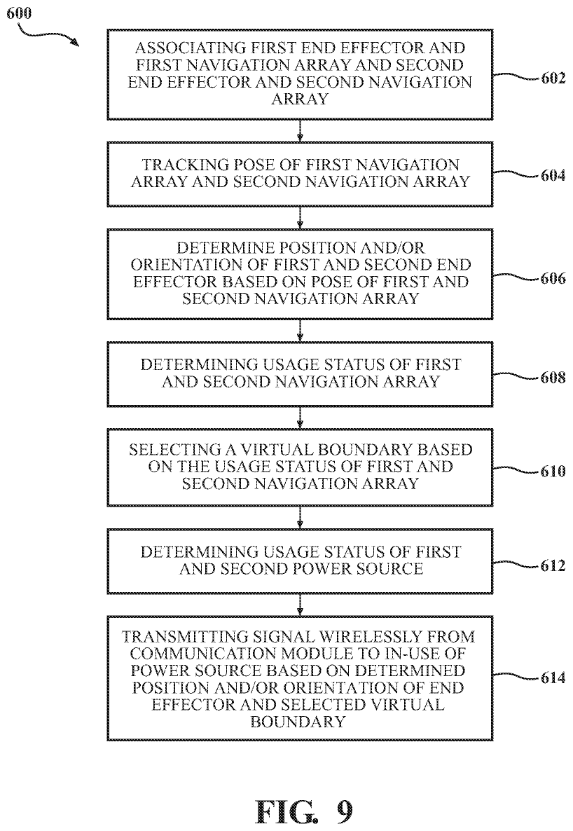

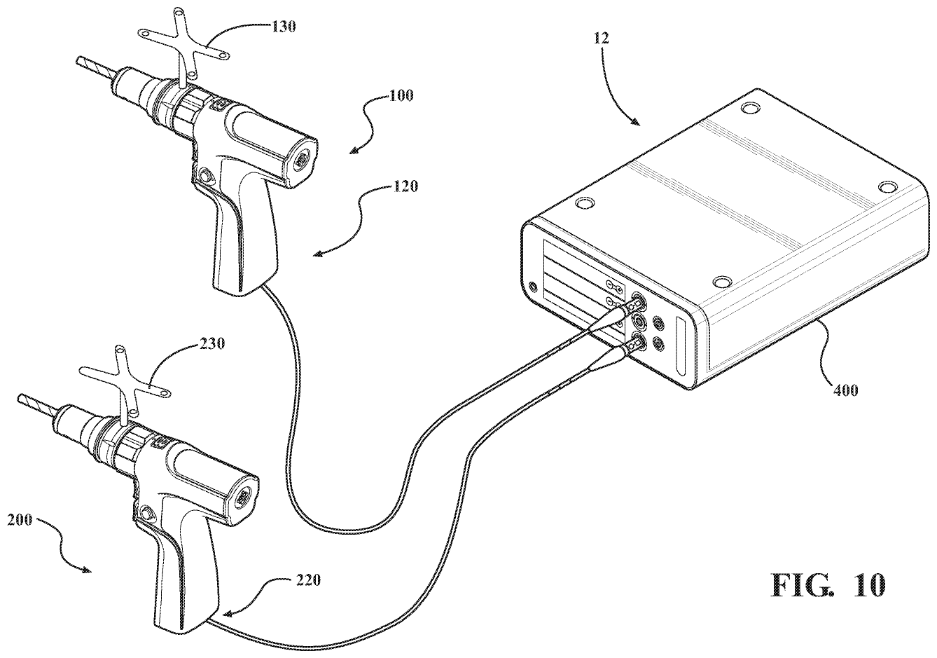

As an alternative or in addition to the battery signal being received by the navigation controller 20 for generating the second association between the power source 160, 260, 360 and the navigation array 130, 230, 330, the power source 160, 260, 360 may include an inertial sensor 170, 270, 370 (only 170 identified) configured to generate a movement signal upon handling of the hand-held surgical tool 120, 220, 320. Referring now to FIG. 7, the first inertial sensor 170 is in electronic communication with the first communication module 165 of the first power source 160. Upon the surgeon handling the first surgical instrument 100, for example, transition between surgical instruments 100, 200, 300 during execution of the surgical plan the movement signal is wirelessly transmitted to the communication module 28. Similar to ease provided the “test pull” implementation above, the surgeon need only handle the hand-held surgical tool 120, 220, 320 for the navigation-assisted surgery system 12 to determine which surgical instrument 100, 200, 300 is “in use.” Other techniques are also contemplated to determine which instrument is in-use and are contemplated as a basis for generating associations between the power sources 160, 260, 360 and the tracking arrays 130, 230, 330 as described herein.

The inertial sensor 170, 270, 370 generating the movement signal may also be used during the presurgical preparation to associate the power source 160, 260, 360, and the navigation array 130, 230, 330 (and/or the end effector 140, 240, 340). The navigation controller 20 receives the movement signal with the movement signal being indicative of the hand-held surgical instrument being handled or manipulated by the user as detected by the inertial sensor 170, 270, 370. The navigation controller 20 generates a third association between the power source 160, 260, 360, and the navigation array 130, 230, 330 based on the movement signal. The third association may be stored in the memory unit 26.

To facilitate the navigation controller 20 generating the third association, the GUI 18 may display prompts 93, 95 to induce the user to handle or manipulate one of the surgical instruments 100, 200, 300. In other words, to ensure that perhaps inadvertent or minor movement of the surgical instrument 100, 200, 300 does not result in an erroneous third correlation, the GUI 18 may display the prompts 93, 95 or instructions. FIG. 7 shows the prompts 93, 95 as arrows indicative of gestures to be mimicked by the user to generate the movement signal. Since the surgical tool 120, 220, 320 is handheld, it is movable in six degrees of freedom—translation along the x-, y- and z-axes and rotation in pitch, yaw, and roll—represented by the directional arrows in FIG. 7. The first prompt 93 instructs the user to move the surgical instrument 100 laterally to the right (translation along the z-axis), and the second prompt 95 instructs the user to rotate the first surgical instrument in roll (rotation about the x-axis). The gestures may be displayed in a predetermined sequence, and the sequence may include one, two, three or four or more gestures in any one or more of the six degrees of freedom. The first inertial sensor 170 senses the movement, and the localizer 22 may also detect the movement with tracking of the first navigation array 130. The movement signal(s) are received by the navigation controller 20 and compared with the gestures displayed on the GUI 18. Should the movement signals match the gestures, the navigation controller 20 generates the third association based on the movement signal. Any and all functionality of the navigation-assisted surgery system 12 described herein may be realized by the present implementation using the inertial sensor 170, 270, 370.

Referring to FIGS. 8 and 9, two alternative workflows 500, 600 are shown. The workflow 500 shown in FIG. 8 illustrates a workflow for controlling a hand-held surgical tool 120, 220, 320. The workflow 600 shown in FIG. 9 illustrates a workflow for controlling a hand-held surgical tool 120, 220, 320 including an in-use power source 160, 260, 360. In workflow 500 and workflow 600 the navigation controller 20 determines an association between the navigation array 130, 230, 330 and the end effector 140, 240, 340 without associating the power source 160, 260, 360. Alternatively, in other workflows, the navigation controller 20 may determine an association between the navigation array 130, 230, 330 and power source 160, 260, 360 without associating the end effector 140, 240, 340.

It should be noted that, while the steps of workflow 500 and workflow 600 are shown in one order, any suitable ordering of the steps may be implemented. Furthermore, it should be appreciated that the various aspects of workflow 500 may be combined with various aspects of workflow 600 and vice-versa.

Workflow 500 includes a step 502 of associating the end effector 140, 240, 340 of a hand-held surgical tool 120, 220, 320 with a navigation array 130, 230, 330. Specifically, the step 502 includes receiving an input indicative of a type of the end effector 140, 240, 340 being coupled to the hand-held surgical tool 120, 220, 320, where the input may be a user input to the GUI 18 that includes a selection of a type of the end effector 140, 240, 340. Step 502 also includes receiving with the navigation controller 20 an identification signal from the localizer 22 detecting the navigation array 130, 230, 330; generating, with the navigation controller 20, an association between the end effector 140, 240, 340 and the navigation array 130, 230, 330 based on the input indicative of a type of the end effector 140, 240, 340 and the identification signal; and storing, with the memory unit 26, the association.

Once the end effector 140, 240, 340 is associated with the navigation array 130, 230, 330, a virtual boundary may be selected during step 504. During step 504, the virtual boundary may be selected such that the virtual boundary is specific to the end effector 140, 240, 340 is associated with the navigation array 130, 230, 330. This ensures that the end effector being used is matched to the boundary specifically designed for that end effector.

Workflow 500 also includes a step 506 of tracking a pose of the navigation array 130, 230, 330 and a step 508 of determining a position and/or an orientation of the end effector 140, 240, 340 based on the pose of the navigation array 130, 230, 330.

During step 510, the communication module 28 receives a battery signal that is wirelessly transmitted from the power source 160, 260, 360 of the hand-held surgical tool 120, 220, 320. The battery signal may be specific to the power source 160, 260, 360 of the hand-held surgical tool 120, 220, 320, such that the navigation controller 20 may determine to which power source 160, 260, 360 to transmit signals. As such, in some cases, the battery signal may be a battery identification signal.

Once the battery signal is received during step 510, the communication module 28 may transmit a signal wirelessly to the power source 160, 260, 360 associated with the battery signal during step 512. Furthermore, once the virtual boundary is selected during step 504, the communication module 28 may transmit the signal to the power source 160, 260, 360 based on the determined position and/or orientation of the end effector 140, 240, 340 (determined during step 508) and the selected virtual boundary. In some instances, the power source 160, 260, 360 may then provide a control signal to a controller of the hand-held surgical tool 120, 220, 320 based on the signal received from the communication module 28 to cause the controller of the hand-held surgical tool 120, 220, 320 to brake the motor 145, 245, 345 of the hand-held surgical tool 120, 220, 320 or otherwise stop or slow the motor of the hand-held surgical tool. In other instances, the signal transmitted wirelessly from the communication module 28 controls a characteristic of energy being supplied from the power source 160, 260, 360 to the hand-held surgical tool 120, 220, 320. For example, the signal transmitted wirelessly from the communication module 28 may result in the power source terminating current being supplied to the first motor 145. This may be implemented using a switch in the power sources.

In some configurations, the workflow 500 may also include determining a pose of a patient tracker coupled to patient anatomy at the surgical site 30 and controlling the hand-held surgical tool 120, 220, 320 based on the position and/or the orientation of the end effector 140, 240, 340 and the pose of the patient tracker. For example, in some instances, controlling the hand-held surgical tool 120, 220, 320 may include providing a control signal to a controller of the hand-held surgical tool 120, 220, 320 to cause the controller of the hand-held surgical tool 120, 220, 320 to brake the motor 145, 245, 345 of the hand-held surgical tool 120, 220, 320. In other instances, controlling the hand-held surgical tool 120, 220, 320 may include controlling a characteristic of energy being supplied from the power source 160, 260, 360 to the hand-held surgical tool 120, 220, 320, such as terminating current being supplied to the first motor 145.

In instances where the navigation-assisted surgery system 12 includes more than one surgical instrument 100, 200, 300, the navigation controller 20 may alter operation of a single hand-held surgical tool 120, 220, 320 or more than one hand-held surgical tool 120, 220, 320. To determine how to alter the operation of a hand-held surgical tool(s) 120, 220, 320, the navigation controller 20 may determine which navigation array 130, 230, 330 and, in some cases, which power source 160, 260, 360 is in use. This process is illustrated in workflow 600, shown in FIG. 9.

It should be noted that, in relation to workflow 600, the first surgical instrument 100 and the second surgical instrument 200, and components thereof, are described below for illustrative purposes only. Workflow 600 may apply to a greater number of surgical instruments and may, alternatively or additionally, apply to the third surgical instrument 300 or any combination of two or more surgical instruments 100, 200, 300.

Workflow 600 may include a step 602 of associating the first end effector 140 with the first navigation array 130 and associating the second end effector 240 with the second navigation array 230. Specifically, the step 602 includes receiving a first input indicative of a type of the first end effector 140 being coupled to the first hand-held surgical tool 120 and a second input indicative of a different type of the second end effector 240 being coupled to the second hand-held surgical tool 220. The first and second input may be a user input to the GUI 18 that includes a selection of a type of the first and second end effector 140, 240. Step 502 also includes receiving, with the navigation controller 20, a first and second identification signal from the localizer 22 detecting the first and second navigation array 130, 230, respectively. Step 502 also includes generating, with the navigation controller 20, a first association between the first end effector 140 and the first navigation array 130 based on the first input and the first identification signal and generating, with the navigation controller 20, a second association between the second end effector 240 and the second navigation array 230 based on the second input and the second identification signal. Step 502 also includes storing, with the memory unit 26, the first and second association.

Workflow 600 also includes a step 604 of tracking a pose of the first and second navigation array 130, 230 and a step 606 of determining a position and/or an orientation of the first and second end effector 140, 240 based on the pose of the first and second navigation array 130, 230, respectively.

Workflow 600 also includes a step 608 of determining a usage status of the first navigation array 130 and the second navigation array 230. Specifically, step 608 may include determining, in a known coordinate system, a pose of the first navigation array 130 and a pose of the second navigation array 230; determining a position and/or orientation of a reference location relative to the known coordinate system; and determining the usage status of the first navigation array 130 and the second navigation array 240 based on the pose of the navigation array 130, the pose of the second navigation array 230, and the position and/or the orientation of the reference location. A reference location may be a point, surface, or volume in the coordinate system used to locate an end effector 140, 240, 340 relative a target state, such as a target object. In one particular implementation, the reference location is a planned axis of a screw. For example, the reference location may be a surface of a bone, a point within a bone, an imaginary or virtual point within the known coordinate system, a volume in the coordinate system, or a combination thereof. The position and/or orientation of the reference location is known with respect to the patient tracker through registration and suitable planning steps.

Once the usage status of the first navigation array 130 and the second navigation array 230 are determined, the navigation controller 20 may select an in-use navigation array 130, 230 and a virtual boundary based corresponding to the in-use navigation array 130, 230 during step 610. The virtual boundary selected may correspond to the in-use navigation array 130, 230.

Workflow may also include a step 612 of determining a usage status of the first power source 160 and the second power source 260 and determining an in-use power source 160, 260 based on the usage status of the first power source 160 and the second power source 260. In some instances, determining the usage status of the first power source 160 and the second power source 260 includes receiving, with the communication module 28, a first and/or second movement signal that is wirelessly transmitted from the first power source 160 and the second power source 260, respectively. The movement signal may be indicative of the first and/or second power source 160, 260 being manipulated by a user as detected by the first and/or second inertial sensor 170, 270, respectively, and that the first and/or power source is/are in-use.

In other instances, determining the usage status of the first power source 160 and the second power source 260 includes receiving, with the communication module 28, a first and/or second battery signal that is wirelessly transmitted from the first and/or second power source 146, 260 during actuation of the first and/or second hand-held surgical tool 120, 220, respectively. The battery signal may be indicative of current being drawn on the first and/or second power source 160, 260, respectively, during a “test-pull” (described previously) and that the first and/or second power source 160, 260 is/are in-use. By confirming whether the power source is in-use at the time of performing the surgery, the system is able to confirm that the battery which will receive the command to control the surgical instrument based one the position and/or orientation of the end effector is the same instrument that the user is actively using, as opposed to being a tool that is being prepared for surgery by one or more operating room technicians, adjacent to the back table or preparation area.

Once the navigation controller 20 selects the virtual boundary and determines the in-use power source 160, 260, the navigation controller 20 may transmit a signal wirelessly to the in-use power source 160, 260 during step 614 based on the virtual boundary and the determined position and/or orientation of the end effector 140, 240 corresponding to the in-use power source 160, 260.

The signal may also and additionally be transmitted wirelessly to the out-of-use power source 160, 260 to cause both/all power sources 160, 260 to stop or to terminate current being supplied to the motor 145, 245. This may be a beneficial approach to risk mitigation in that all instruments being tracked by the localizer are ceased so that if one of the end effectors was switched to a different array without providing the appropriate input signal to the navigation system, the navigation system would nonetheless stop the instrument from penetrating beyond the virtual boundary. In some instances, the navigation controller 20 may determine that a power source 160, 260 is in-use based on a time of receiving a movement signal and/or a battery signal and transmit a signal wirelessly to the in-use power source 160, 260 and out-of-use power source 160, 260 based on the time of receiving the movement signal and/or the battery signal. As an example, in an instance where the first switch 150 is engaged before the second switch 250, the navigation controller 20 may determine that the first hand-held surgical tool 120 is out-of-use and that the second hand-held surgical tool 220 is in-use (or vice versa). The navigation controller 20 may then transmit the signal to the second hand-held surgical tool 220 before the first hand-held surgical tool 120. This utilization of the time of receiving the battery signal and/or movement signal ensures that the latency of communication between the in-use power source and navigation system is minimized and/or prioritized relative to the latency of communication between the out-of-use power source and the navigation system. In other words, the in-use energy source will receive the signal from the navigation system before the out-of-use power source.

For ease of use with executing the surgical plan, it may be desirable for each one of the surgical instruments 100, 200, 300 to include its own navigation array 130, 230, 330, end effector 140, 240, 340, and power source 160, 260, 360. FIGS. 1 and 2, for example, show three sets of components for three surgical instruments 100, 200, 300. As mentioned, however, it may be indicated to reestablish the associations during execution of the surgical plan, or perhaps during presurgical preparation. For examples, one of the power sources 160, 260, 360 may use all of its charge, or one of the communication modules 165, 265, 365 may lose two-way wireless communication with the communication module 28 of the navigation-assisted surgery system 12. The power source 160, 260, 360 may be interchangeably moved between the hand-held surgical tools 120, 220, 320, and the first, second, and/or third associations may be reestablished. The user may perform the workflow previously described by engaging the GUI 18 to identify the navigation array 130, 230, 330 coupled to the hand-held surgical tool 120, 220, 320 to which the power source 160, 260, 360 being moved is now coupled. The updated second association or updated third association based on the battery signal and the movement signal, respectively, is updated and stored in the memory unit 26. Any aspect of the workflow may also be used to reestablish the associations.

Additionally, it is contemplated that the surgical instruments 100, 200, 300 may be coupled to a console. As shown in FIG. 10, the navigation-assisted surgery system 12 includes a console 400, which may be coupled to the surgical instruments 100, 200, 300 using a cable. In FIG. 10, the console 400 is shown coupled to the first surgical instrument 100 and the second surgical instrument 200. The first surgical instrument 100 includes the first hand-held surgical tool 120 coupled to the first navigation array 130 and the second surgical instrument 200 includes the second hand-held surgical tool 220 coupled to the second navigation array 230. It should be noted that the console 400 may be coupled to any suitable number of surgical instruments 100, 200, 300. For example, the console 400 may be coupled to one surgical instrument, or more than two surgical instruments.

In instances where the navigation-assisted surgery system 12 includes a console 400, the console 400 may transmit signals to the first hand-held surgical tool 120 and the second hand-held surgical tool 220 to control operation of the first hand-held surgical tool 120 and the second hand-held surgical tool 220. The console may determine which of the first and second hand-held surgical tools are in use based on which ports of the console have current being drawn therethrough or which motor controllers are active within the console. For example, the console 400 may transmit a signal to the in-use hand-held surgical tool to cease operation based on a position and/or orientation of the end effector associated with the in-use navigation array and based on the virtual boundary. As another example, the console 400 may transmit a signal to the in-use hand-held surgical tool and to the out-of-use hand-held surgical tool to cease operation of both the in-use hand-held surgical tool and the out-of-use hand-held surgical tool.

The foregoing description is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described. It should be appreciated that any structure, function, and/or workflow steps described with reference to the first surgical instrument 100, or any of its components, may be included or performed on the second surgical instrument 200, the third surgical instrument 300, or any additional surgical instruments.

1.-84. (canceled)

85. A method of operating a navigation-assisted surgery system including a hand-held surgical tool, an end effector coupled to the hand-held surgical tool and a navigation array coupled to the hand-held surgical tool or the end effector, a power source coupled to the hand-held surgical tool, a localizer, a communication module, a memory unit, and a navigation controller, the method comprising:
receiving with an input indicative of a type of the end effector coupled to the hand-held surgical tool;
receiving with the navigation controller an identification of the hand-held surgical tool;
receiving with the navigation controller a movement signal that is wirelessly transmitted from the communication module, the movement signal being indicative of the hand-held surgical tool being manipulated by a user; and
generating with the navigation controller an association between the hand-held surgical tool and one of the navigation array and the end effector based on the movement signal; and
storing with the memory unit the association.

86. The method of claim 85, wherein the navigation-assisted surgery system includes a display, and further comprising displaying, with the display, gestures to be mimicked by movements of the hand-held surgical tool from the user to generate the movement signal.

87. The method of claim 86, further comprising:
displaying the gestures in a predetermined sequence; and
comparing a sequence of the movements of the hand-held surgical tool with the predetermined sequence.

88. The method of claim 87, wherein the gestures are at least one of movement in translation and movement in rotation.

89. The method of claim 88, wherein the movement in rotation is rotation in at least one of pitch, yaw, and roll.

90. The method of claim 86, further comprising displaying on the display an aspect of a surgical plan associated with the type of the end effector.

91. The method of claim 85, further comprising correlating the type of the end effector with an aspect of a surgical plan including a predefined virtual boundary that is based on the type of the end effector.

92. The method of claim 91, further comprising:
tracking a pose of the navigation array;
determining a position and/or an orientation of the end effector based on the pose of the navigation array; and
transmitting a control signal wirelessly from the navigation controller to the communication module to control a characteristic of energy being supplied to the hand-held surgical tool based on the position and/or the orientation of the end effector relative to the predefined virtual boundary.

93. The method of claim 92, wherein the control signal is configured to terminate current being supplied when the position and/or the orientation of the end effector crosses the predefined virtual boundary.

94. The method of claim 85, further comprising receiving with the navigation controller a battery signal that is wirelessly transmitted from the communication module during actuation of the hand-held surgical tool, the battery signal being indicative of current being drawn on the power source.

95. The method of claim 85, wherein the navigation-assisted surgery system includes a user interface, and further comprising receiving with the user interface a user input including a selection of the type of the end effector.

96. The method of claim 85, wherein the navigation-assisted surgery system includes a camera, and further comprising detecting with the camera a characteristic of the end effector indicative of the type of the end effector coupled to the hand-held surgical tool.

97. A navigation-assisted system for controlling a hand-held surgical tool to which each of an end effector and a navigation array is coupled, the system comprising:
a sensor configured to detect manipulation of the hand-held surgical tool and generate a movement signal in response to detecting manipulation of the hand-held surgical tool;
a communication module in communication with the sensor and being configured to transmit the movement signal;
a localizer configured to generate an identification in response to detecting the navigation array; and
a navigation controller being configured to:
receive the identification signal from the localizer;
receive the movement signal from the communication module; and
generate and store an association between the hand-held surgical tool and one of the navigation array and the end effector based on the movement signal.

98. The navigation-assisted system of claim 97, further comprising a user interface configured to receive an input indicative of a type of the end effector coupled to the hand-held surgical tool.

99. The navigation-assisted system of claim 97, further comprising a camera configured to detect a characteristic of the end effector indicative of a type of the end effector coupled to the hand-held surgical tool.

100. A navigation-assisted system for wirelessly controlling a first hand-held surgical tool to which each of a first end effector, a first navigation array, and a first power source is coupled and a second hand-held surgical tool to which each of a second end effector, a second navigation array, and a second power source is coupled, the system comprising:
a localizer comprising a sensor configured to detect the navigation array and the second navigation array;
a navigation controller configured to:
receive a first input indicative of a type of the first end effector coupled to the first hand-held surgical tool;
receive a second input indicative of a type of the second end effector coupled to the second hand-held surgical tool;
receive a first identification signal from the first navigation array;
receive a second identification signal from the second navigation array;
generate a first association between the first end effector and the first navigation array based on the first input and the first identification signal;
generate a second association between the second end effector and the second navigation array based on the second input and the second identification signal;
determine a usage status of the navigation array and the second navigation array;
select an in-use navigation array among the first navigation array and the second navigation array based on the usage status of the first navigation array and the usage status of the second navigation array;
select a virtual boundary based on the identification signal received from the in-use navigation array;
determine a usage status of the first hand-held surgical tool and the second hand-held surgical tool;
select an in-use hand-held surgical tool among the first hand-held surgical tool and the second hand-held surgical tool based on the usage status; and
transmit a signal to the in-use hand-held surgical tool to cease operation based on a position and/or orientation of the end effector associated with the in-use navigation array and based on the selected virtual boundary.

101. The navigation-assisted system of claim 100, wherein the navigation controller is further configured to:
determine an out-of-use hand-held surgical tool among the first hand-held surgical tool and the second hand-held surgical tool based on the usage status of the first hand-held surgical tool and the usage status of the second hand-held surgical tool; and
transmit a second signal to the out-of-use hand-held surgical tool based on the position of the end effector and the position of the selected virtual boundary.

102. The navigation-assisted system of claim 100, wherein the hand-held surgical tool and the second hand-held surgical tool each include a hand-held surgical tool controller, and wherein the signal transmitted from the navigation controller causes the hand-held surgical tool controller of the in-use hand-held surgical tool to brake a motor of the in-use hand-held surgical tool.

103. The navigation-assisted system of claim 100, further comprising a user interface configured to receive an input indicative of a type of the end effector coupled to the first hand-held surgical tool.

104. The navigation-assisted system of claim 100, further comprising a camera configured to detect a characteristic of the end effector indicative of the type of the end effector coupled to the hand-held surgical tool.
